SEQUENCE LISTING (5 sequences)

SEQ ID NO: 1 - RNA, 80 nt, synthetic construct
  source 1..80: mol_type = other RNA
gttttagagctagaaatagcaagttaaaataaggctagtccgttatcaacttgaaaaagtggcaccgagtcggtgctttt

SEQ ID NO: 2 - DNA, 23 bp, synthetic construct
  source 1..23: mol_type = other DNA
gttttagagctagaaatagcaag

SEQ ID NO: 3 - DNA, 18 bp, synthetic construct
  source 1..18: mol_type = other DNA
cggtgccactttttcaag

SEQ ID NO: 4 - DNA, 26 bp, synthetic construct
  source 1..26: mol_type = other DNA
ttaaaataaggctagtccgttatcaa

SEQ ID NO: 5 - DNA, 17 bp, synthetic construct
  source 1..17: mol_type = other DNA
aaagcaccgactcggtg

(19) United States
(12) Patent Application Publication
(10) Pub. No.: US 2024/0417794 A1
(43) Pub. Date: Dec. 19, 2024

(54) PRIMERS, KIT AND METHOD FOR DETECTING RESIDUAL AMOUNT OF SGRNA IN ENVIRONMENT

(71) Applicant: NANJING GENSCRIPT BIOTECH CO., LTD., Nanjing, Jiangsu (CN)

(72) Inventors: Chun GUO, Nanjing (CN); Jingping WU, Nanjing (CN); Ming YAO, Nanjing (CN); Jianpeng WANG, Nanjing (CN); Hong LI, Nanjing (CN)

(73) Assignee: NANJING GENSCRIPT BIOTECH CO., LTD., Nanjing, Jiangsu (CN)

(21) Appl. No.: 18/704,492

(22) PCT Filed: Nov. 3, 2022
(86) PCT No.: PCT/CN2022/129518

§ 371 Date: Apr. 25, 2024

(30) Foreign Application Priority Data
Nov. 3, 2021 (CN) 202111295450.4

(51) Int. Cl. C12Q 1/6876

(52) U.S. Cl. CPC C12Q 1/6876

(57) ABSTRACT

A primer pair, a kit and a method for detecting the residual amount of sgRNA in the environment. The forward primer of the primer pair is a nucleic acid molecule as shown in SEQ ID NO: 2, and the reverse primer of the primer pair is a nucleic acid molecule as shown in SEQ ID NO: 5. The primer pair can be used for detecting the residual amount of sgRNA in the environment; can perform real-time and high-throughput detection on the residual amount of sgRNA in the environment; has the advantages of good specificity, high sensitivity and repeatability, and convenient operation; and can perform real-time and high-throughput detection on the residual amount of the sgRNA in the environment in different stages of a production process.

### CROSS-REFERENCE TO RELATED APPLICATION

The present application claims priority to Chinese patent application No. 202111295450.4, filed on Nov. 3, 2021, which is incorporated herein by reference in its entirety.

### TECHNICAL FIELD

The present invention relates to the technical field of biological detection, and particularly relates to a primer, a kit and a method for detecting the residual amount of sgRNA in the environment.

### BACKGROUND

A CRISPR/Cas system is an acquired immune system that exists in most archaea and bacteria, is used to resist foreign genetic materials (such as plasmids and phages), and leaves foreign gene fragments in its own loci as “memory” to resist their reinvasion. At present, the CRISPR/Cas system has been modified by scientists as a powerful tool for gene editing, and performs genome editing relying on a single guide RNA (i.e., an sgRNA) and a Cas enzyme. The genome targeting specificity depends on the small guide RNA (sgRNA), and the genome targeting sequence has a triple base prototypic adjacent motif (PAM) at the 3′-terminal or the 5′-terminal.

In recent years, as the CRISPR/Cas system has been widely used in the field of cell editing, sgRNA products as key raw materials must meet strict quality standards. However, in an actual production process, there is a possibility of occurrence of cross-contamination due to collinear production, that is, residues of the sgRNA of a last batch in the environment would affect the quality of products of a next batch. According to the guiding principle of the Guidance on Aspects of Cleaning Validation in Active Pharmaceutical Ingredient Plants (APIC): during validation, the company needs to prove that the cleaning procedures used daily for each device can limit potential residues carried into the next product to an acceptable level. The established limits must be calculated scientifically and rationally. If a corresponding residual amount exceeds the limit, it may cause off-target effects in editing, thereby resulting in unpredictable impacts.

Traditional residue detection methods, such as TOC determination, are not specific, and the results cannot directly reflect the residue of the sgRNA. The NGS sequencing method is theoretically feasible, but has long experimental cycle and high costs; its results are closely related to the quality of database construction, and its application in routine environmental monitoring also has its limitations. Therefore, the method for detecting sgRNA residue in the environment remains to be further researched.

### SUMMARY

According to an aspect of the present application, a primer pair for detecting a residual amount of a small guide RNA (sgRNA) in an environment is provided, wherein, a forward primer of the primer pair is a nucleic acid molecule as shown in SEQ ID NO: 2, and a reverse primer of the primer pair is a nucleic acid molecule as shown in SEQ ID NO: 5.

In some embodiments, the sgRNA comprises a sequence as shown in SEQ ID NO: 1.

According to another aspect of the present application, a kit for detecting a residual amount of an sgRNA in an environment is provided. The kit comprises the above-mentioned primer pair.

In some embodiments, the kit further comprises a fluorescent probe, the fluorescent probe is a nucleic acid molecule as shown in SEQ ID NO: 4 with a fluorescent group connected at 5′-terminal of the nucleic acid molecule and a quenching group connected at 3′-terminal of the nucleic acid molecule.

In some embodiments, the fluorescent group comprises FAM, JOE, JA270, TET, Cal Fluor Gold 540, HEX, VIC, Cal Fluor Orang 560, TAMRA, Cyanine 3, Quasar 570, Cal Fluor Red 590, Rox, Texas Red, Cyanine 5, Quasar 670, or Cyanine 5.5; the quenching group comprises MGB, TAMRA, DABCYL, BHQ1-3, or Eclipse.

In some embodiments, the sgRNA comprises a sequence as shown in SEQ ID NO: 1.

In some embodiments, the kit is used in a real-time fluorescence quantitative PCR system.

According to still another aspect of the present application, a method for detecting a residual amount of an sgRNA in an environment is provided. the method comprises:
1) designing a primer pair and a fluorescent probe based on a sequence of a residual sgRNA in the environment;
2) locally sampling a region in the environment to obtain a sample;
3) performing real-time fluorescence quantitative PCR on the sample obtained from the local sampling using the primer pair and the probe in step 1), and determining a concentration of the sgRNA in the sample obtained from the local sampling using a Ct value; and
4) determining the residual amount of the sgRNA in the environment based on the concentration of the sgRNA in the sample determined in step 3).

In some embodiments, the sgRNA comprises the sequence as shown in SEQ ID NO: 1.

In some embodiments, a forward primer of the primer pair is a nucleic acid molecule as shown in SEQ ID NO: 2, and a reverse primer of the primer pair is a nucleic acid molecule as shown in SEQ ID NO: 5.

In some embodiments, the fluorescent probe is a nucleic acid molecule with a fluorescent group connected at the 5′-terminal of the nucleic acid molecule and a quenching group connected at the 3′-terminal of the nucleic acid molecule.

In some embodiments, the fluorescent probe comprises a nucleic acid molecule as shown in SEQ ID NO: 4.

In some embodiments, the determining the residual amount of the sgRNA in the environment based on the concentration of the sgRNA in the sample comprises: determining a content of the sgRNA in the sample based on the concentration of the sgRNA in the sample; and determining the residual amount of the sgRNA in the environment based on the content of the sgRNA in the sample.

In some embodiments, the fluorescent group comprises FAM, JOE, JA270, TET, Cal Fluor Gold 540, HEX, VIC, Cal Fluor Orang 560, TAMRA, Cyanine 3, Quasar 570, Cal Fluor Red 590, Rox, Texas Red, Cyanine 5, Quasar 670, or Cyanine 5.5; the quenching group comprises MGB, TAMRA, DABCYL, BHQ1-3, or Eclipse.

### BRIEF DESCRIPTION OF THE DRAWINGS

This specification will be further illustrated by way of exemplary embodiments, and these exemplary embodiments will be described in detail with reference to the accompanying drawings. These embodiments are not restrictive, and in these embodiments, the same numerals represent the same structures.

FIG. 1 shows a schematic diagram of two groups of primer probes designed for a universal sgRNA sequence.

FIG. 2 shows an amplification curve of a first group of primer-probe combination FR1P designed for a universal sgRNA sequence.

FIG. 3 shows an amplification curve of a second group of primer-probe combination FR2P designed for a universal sgRNA sequence.

FIG. 4 shows a standard curve diagram plotted after real-time fluorescence quantitative PCR is performed on a reference sgRNA.

FIG. 5 shows a diagram of comparison among residual amount results of an sgRNA in an environment before and after clearance at different stages.

### DETAILED DESCRIPTION

For a clearer description of the technical solutions in the embodiments of the present application, a brief introduction will be given below for the accompanying drawings required for describing the embodiments. Apparently, the accompanying drawings in the following description show merely some embodiments of the present application, and those of ordinary skill in the art may still apply the present application to other similar scenarios according to these accompanying drawings without any creative effort. Unless obvious from the linguistic context or otherwise stated, the same reference signs in the drawings represent the same structure or operation.

As shown in the present application and the claims, the words “one”, “a”, “an” and/or “the” do not specifically refer to the singular, but may also include the plural, unless the context clearly indicates otherwise. Generally, the terms “including” and “comprising” only imply the inclusion of explicitly identified steps and elements, and these steps or elements do not constitute an exclusive list. A method or a device may further comprise other steps or elements.

In recent years, due to unique advantages in terms of specificity, sensitivity, and accuracy, the fluorescent probe detection technology has been widely recognized and applied in disease-related detection fields such as detection of gene mutation, gene quantification, etc. Fluorescent probe detection belongs to real-time fluorescence quantitative PCR technology, and is a rapid high-throughput detection method. A specific fluorescent probe is added during a PCR amplification process to reflect the increase of the product through fluorescence signal changes, thereby enabling quantitative analysis of an initial template in the sample.

In the present application, the real-time fluorescent quantitative PCR technology (RT-qPCR) based on the fluorescent probe is used to design a specific primer and a fluorescent probe for a universal sgRNA sequence, thereby performing real-time and high-throughput detection of the residual amount of the sgRNA in the environment with advantages of good specificity, high sensitivity, high repeatability, and convenient operations, and performing real-time and high-throughput detection of the residual amount of the sgRNA in the environment at different stages of the production process.

According to an aspect of the present application, a method for detecting a residual amount of an sgRNA in an environment is provided.

In some embodiments, the environment may be a production environment involving the sgRNA, for example, a production workshop where biological products (e.g., plasmids, etc.) are produced using the sgRNA as a raw material. In some embodiments, the environment may be other environments, for example, an environment where products involving the sgRNA are sold, an environment where products involving the sgRNA are shipped, etc.

In order to detect the sgRNA residue in the environment, local sampling can be performed on a region in the environment (also referred as a to-be-sampled region), that is, sampling is performed on a preset region (also referred to as a local region) selected on the to-be-sampled region in the environment to obtain a sample after local sampling (also referred to as a sample of local sampling). The to-be-sampled region is a region where there may be sgRNA residue present. The local region is a region where local sampling is performed on the to-be-sampled region. Taking the production environment as an example, the to-be-sampled region may include wall surfaces, floors, roofs, operating tables, shelves, and devices involving the sgRNA (for example, a synthesizer, a freeze dryer, a pallet, or a PCR instrument). For example only, the to-be-sampled region is a wall surface, and the local region is a part of the wall surface where sampling is performed. By locally sampling of a region in the environment, the residual amount of the sgRNA in the environment can be estimated. For example, the residual amounts of the sgRNA in local regions can be summed to obtain the residual amount of the sgRNA in the region of the environment, and then the residual amount of the sgRNA in the environment can be obtained based on the residual amount of the sgRNA in the region of the environment. Taking a shipping environment of products involving the sgRNA as an example, the to-be-sampled region may include a packaging region of to-be-shipped products, a surface region of the to-be-shipped products, a loading region, etc. By sampling a local region in the to-be-sampled region of the shipping environment, the residual amount of the sgRNA in the shipping environment can be determined, and then whether the to-be-shipped products are contaminated can be determined. It should be noted that the sgRNA residue can also be determined by detecting only a part of contents in the environment, for example, only to-be-shipped products are detected, only a device is detected, etc.

It should be noted that the sampling method of regions in the environment is not limited in the present application. For example, in the present application, overall sampling can be performed on the regions in the environment to compute the residual amount of the sgRNA in the regions, and then the residual amount of the sgRNA in the environment may be determined.

In some embodiments, after the production department completes a production, clearance would be performed on the environment (for example, the production clean room), and the residual amount of the sgRNA in the environment after clearance can be detected using the method of the present application. Before clearance, collection of the sample of local sampling is also performed to detect the residual amount of the sgRNA in the environment before clearance, to test the clearance effects.

In some embodiments, a local region in the environment may be sampled through a sampling method. Exemplary sampling methods may include a swabbing method, a contact disc method, a surface rinsing method, etc. Taking the swabbing method as an example, a sterilized disposable medical swab is wetted by dipping in an EP tube containing a certain volume (for example, 500 ul) of a sterilized and nuclease-free solution (for example, water, physiological saline, etc.). A preset region of the to-be-sampled region is selected and wiped back and forth. After the sampling is complete, the swab is placed into a corresponding EP tube, and broken. Then, the tube is covered with the tube cap. At this point, the collection of a sample of local sampling is completed. The preset region may be a region of any size selected on the to-be-sampled region, for example, 10 cm×10 cm, 20 cm×20 cm, 10 cm×20 cm, etc. The residual amount of the sgRNA in the whole to-be-sampled region is estimated based on the residual amount of the sgRNA in the preset region. In particular, by sampling on the preset region of the to-be-sampled region, the residual amount of the sgRNA in the preset region is determined, thereby estimating the residual amount of the sgRNA in the whole to-be-sampled region. In some embodiments, the residual amount of the sgRNA in the to-be-sampled region can be determined based on the residual amount of the sgRNA in the preset region (i.e., the sampling amount of the collected sample of local sampling) and an area ratio of the to-be-sampled region to the preset region, for example, determined by multiplying the sampling amount of the collected sample of local sampling by the area ratio of the to-be-sampled region to the preset region.

In some embodiments, a primer pair and a fluorescent probe can be designed based on a sequence of a residual sgRNA in the environment to detect the residual amount of the residual sgRNA during the production or experiment process, to avoid possible subsequent cross-contamination. In particular, the universal sgRNA sequence can be inputted into SnapGene software to design a plurality of pairs of upstream and downstream primers and probe sequences. Real-time fluorescence quantitative PCR experiments are then performed to validate the effects of the combinations of the plurality of pairs of primers and the probes. The “design and preferred selection of qPCR primers and probes” section in the Examples may be referred to for details. It should be noted that the detection method of the present application can be applied to various residual sgRNAs in the environment, and this is not limited in the present application.

In some embodiments, the content of the sgRNA in the sample obtained from local sampling can be determined through real-time fluorescence quantitative PCR technology using the determined sgRNA primer pair and probe. In particular, the content of the sgRNA in the sample can be determined by determining the Ct value. The sgRNA comprises a universal sequence and a specific sequence, and primers and probes can be designed based on the universal sequence. In some embodiments, the sgRNA comprises a universal sequence as shown in SEQ ID NO: 1. In some embodiments, a plurality of pairs of primers and probes (for example, 2 pairs) can be designed for the universal sgRNA sequence, and the effects of the designed primers and fluorescent probes can be validated through the real-time fluorescence quantitative PCR experiments, for example, by ascertaining whether the amplification efficiency satisfies conditions and whether the negative control results are normal.

In some embodiments, a primer pair designed for the universal sgRNA sequence comprises a forward primer as shown in SEQ ID NO: 2 and a reverse primer as shown in as SEQ ID NO: 5. In some embodiments, a fluorescent probe designed for the universal sgRNA sequence is as shown in SEQ ID NO: 4, the 5′-terminal of the fluorescent probe can be connected to a fluorescent group, and the 3′-terminal of the fluorescent probe can be connected to a quenching group. In some embodiments, the fluorescent group may include, but is not limited to, FAM, JOE, JA270, TET, Cal Fluor Gold 540, HEX, VIC, Cal Fluor Orang 560, TAMRA, Cyanine 3, Quasar 570, Cal Fluor Red 590, Rox, Texas Red, Cyanine 5, Quasar 670, and Cyanine 5.5. In some embodiments, the 5′-terminal of the fluorescent probe may be connected to FAM. In some embodiments, the quenching group may include, but is not limited to, MGB, TAMRA, DABCYL, BHQ1-3, and Eclipse. In some embodiments, the 3′-terminal of the fluorescent probe may be connected to MGB.

In some embodiments, the concentration of the sgRNA in the collected sample of local sampling can be determined from a standard curve. The standard curve can be determined by performing a series of dilutions of a reference sgRNA and then performing real-time fluorescence quantitative PCR.

The content of the sgRNA in the sample of local sampling can be determined by determining the concentration of the sgRNA in the collected sample of local sampling and the solution volume in the EP tube, that is, the content of the sgRNA in the sample of local sampling collected on the preset region of the to-be-sampled region can be determined according to formula (1).

$$A = C \times V \qquad ( 1 )$$

A represents the content of the sgRNA in the sample of local sampling, C represents the concentration of the corresponding sample of local sampling, and V represents the volume of the sampling solution in the EP tube.

The residual amount of the sgRNA in the to-be-sampled region can be determined based on the content of the sgRNA in the sample of local sampling and the area ratio of the to-be-sampled region to the preset region according to formula (2).

$$S = A \times S 2 / S 1 \qquad ( 2 )$$

S represents the residual amount of the sgRNA in the to-be-sampled region, A represents the content of the sgRNA in the sample of local sampling collected on the preset region of the to-be-sampled region, S2 represents the area of the to-be-sampled region, and S1 represents the area of the preset region of the to-be-sampled region.

Through the above method, the residual amount of the sgRNA in each to-be-sampled region in the environment can be sequentially determined, and can be summed to determine the residual amount of the sgRNA in the environment.

In some embodiments, the residual amount of the sgRNA in the environment can be compared with a preset threshold to determine whether the content of the sgRNA in the environment has an impact on downstream biological applications. For example, the residual amount of the sgRNA detected after clearance of the production workshop is compared with the preset threshold (for example, 20 ug). When the residual amount of the sgRNA is less than 20 ug, it means that there is basically no impact on the downstream biological applications.

According to another aspect of the present application, a primer pair for detecting a residual amount of a small guide RNA (sgRNA) in an environment is provided. The primer pair is used to specifically identify a universal sgRNA sequence, and the universal sgRNA sequence is as shown in SEQ ID NO: 1. A forward primer of the primer pair is a nucleic acid molecule as shown in SEQ ID NO: 2, and a reverse primer of the primer pair is a nucleic acid molecule as shown in SEQ ID NO: 5.

Real-time fluorescence quantitative PCR is performed using the above-mentioned primer pair. Compared with other primer pairs, the Ct value and amplification curve show normal performance, and the Ct values of the negative control are all larger than 40, satisfying the broad acceptable standard in the field of molecular diagnosis.

According to still another aspect of the present application, a kit for detecting a residual amount of an sgRNA in an environment is provided. In some embodiments, the sgRNA may comprise a sequence as shown in SEQ ID NO: 1. The kit may comprise a primer pair for detecting a residual amount of a small guide RNA (sgRNA) in an environment as described above. The kit may further comprise a fluorescent probe, the fluorescent probe is a nucleic acid molecule as shown in SEQ ID NO: 4 with a fluorescent group connected at 5′-terminal of the nucleic acid molecule and a quenching group connected at 3′-terminal of the nucleic acid molecule. In some embodiments, the fluorescent group may include, but is not limited to, FAM, JOE, JA270, TET, Cal Fluor Gold 540, HEX, VIC, Cal Fluor Orang 560, TAMRA, Cyanine 3, Quasar 570, Cal Fluor Red 590, Rox, Texas Red, Cyanine 5, Quasar 670, Cyanine 5.5, etc. In some embodiments, the 5′-terminal of the fluorescent probe may be connected to FAM. In some embodiments, the quenching group may include, but is not limited to, MGB, TAMRA, DABCYL, BHQ1-3, Eclipse, etc. In some embodiments, the 3′-terminal of the fluorescent probe may be connected to MGB. In some embodiments, the fluorescent group is FAM. In some embodiments, the quenching group is MGB.

This kit can be used in a real-time fluorescence quantitative PCR system. In some embodiments, the kit may include a reagent for performing real-time fluorescence quantitative PCR, such as an enzyme solution, a dye, a reaction buffer, etc. In some embodiments, the kit may comprise a reagent for performing one-step real-time fluorescence quantitative PCR, such as a one-step reaction mix, a one-step enzyme mix, a ROX dye, etc.

### EXAMPLES

The experimental methods in the following examples are all conventional methods, unless otherwise specified. The experimental materials used in the following examples were all purchased from conventional biochemical reagent companies, unless otherwise specified. The quantitative experiments in the following examples were performed in triplicate, and the results were averaged.

### Design and Preferred Selection of qPCR Primers and Probes

Based on a universal sequence of a produced sgRNA sequence, two groups of primer-probe combinations FR1P and FR2P were designed. The primers and probes were specifically designed as follows:

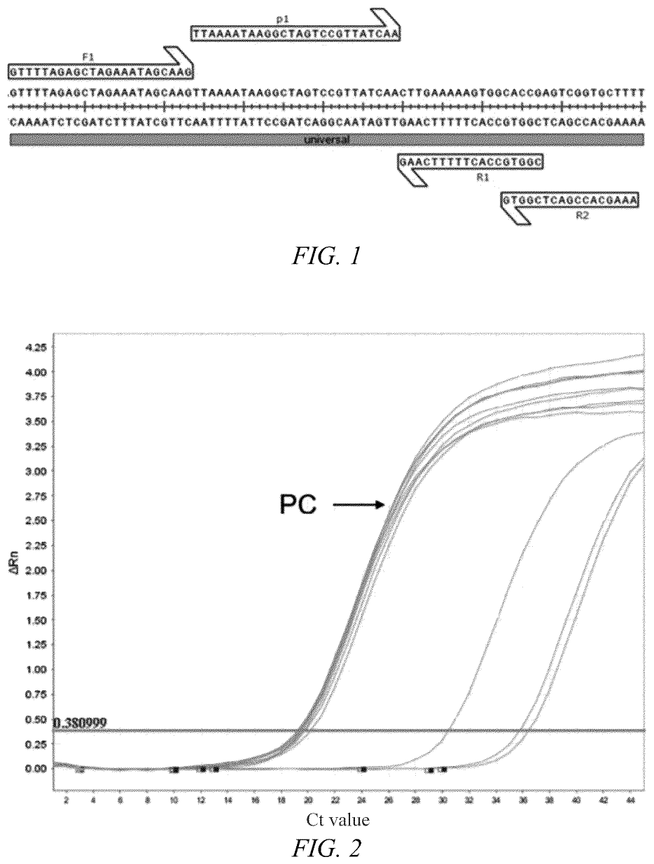

First, the universal sgRNA sequence was inputted into SnapGene software, and a sequence that is complementary paring to the sgRNA sequence can be obtained therefrom. As shown in FIG. 1, an upstream primer F1 and downstream primers R1 and R2 were designed based on the sgRNA sequence. The upstream primer F1 can identify the complementary sequence of the universal sgRNA sequence, and the downstream primers R1and R2 can specifically bind to the universal sgRNA sequence, while ensuring that the sequence length is between 18-24 nt and the primer Tm value is 50-60° C. In addition, the probe was designed in the rear of the upstream primer F1, with the sequence length between 20-35 nt, and with the Tm value of the probe being 8-10° C. higher than that of the upstream and downstream primers. It should be pointed out that the Tm value of the probe is low due to the short fragments available for design, and the Tm value of the probe is increased using MGB modification combination to achieve the purpose of specific binding of the probe to a target sequence, which is beneficial to better amplification. In particular, the probe is connected to a MGB (Minor Groove Binder) modification group, which can increase the Tm value of the probe by about 10° C. Therefore, the MGB probe can be designed to be shorter than an ordinary TaqMan probe. A non-fluorescent quencher is used as a quencher of the MGB probe, the non-fluorescent quencher itself does not produce fluorescence, and can greatly reduce the intensity of the background signal. Here, FAM and MGB are connected to either terminal of the probe P respectively. The two groups of designed primer-probe combinations FR1P and FR2P are as shown in Tables 1 and 2 below. In the two groups, the sequences of the upstream primers are the same and the sequences of the probes are the same, but the sequences of the downstream primers are different. It should be noted that the probes can specifically recognize the universal sgRNA sequence, and can also specifically recognize the complementary sequence of the universal sgRNA sequence. FIG. 1 merely shows specific recognization of the complementary sequence of the universal sgRNA sequence, but this should not be used as a limitation on the present application.

TABLE 1
Primer-probe combination FR1P

| | | Modifi- | |
|---|---|---|---|
| Name | Sequence | cation | Tm |
| F1 | GTTTTAGAGCTAGAAATAGCAAG | / | 50 |
| | (SEQ ID NO: 2) | | |
| R1 | CGGTGCCACTTTTTCAAG | / | 53 |
| | (SEQ ID NO: 3) | | |
| P | TTAAAATAAGGCTAGTCCGTTAT | 5′-FAM; | 52 |
| | CAA (SEQ ID NO: 4) | 3′-MGB | |

TABLE 2
Primer-probe combination FR2P

| | | Modifi- | |
|---|---|---|---|
| Name | Sequence | cation | Tm |
| F1 | GTTTTAGAGCTAGAAATAGCAAG | / | 50 |
| | (SEQ ID NO: 2) | | |
| R2 | AAAGCACCGACTCGGTG | / | 55 |
| | (SEQ ID NO: 5) | | |
| P | TTAAAATAAGGCTAGTCCGTTAT | 5′-FAM; | 52 |
| | CAA (SEQ ID NO: 4) | 3′-MGB | |

Real-time fluorescence quantitative PCR experiments were performed on a positive control (PC, synthesized sgRNA sample at a known concentration) and a no template control (NTC) using the above-mentioned two groups of primer-probe combinations FR1P and FR2P, to validate the effects of the two groups of primer-probe combinations. The positive control was sgRNA samples of PC-101, PC-201, and PC-001 containing the universal sequence SEQ ID NO: 1 in Table 3, and the concentrations were all 0.1 pg/ul. Each group was provided with 3 positive controls and a no template control respectively, and each control was provided with triplicate wells respectively. After the experiment was complete, the amplification curve and the Ct value of the sample were obtained, to select a preferred combination satisfying the conditions, such as amplification efficiency and normal negative control results.

The results of real-time fluorescence quantitative PCR are as shown in Table 3 below.

TABLE 3
qPCR results of FR1P and FR2P

| Primer-probe | | Sample | | | |
|---|---|---|---|---|---|
| combination | Ct value | PC-101 | PC-201 | PC-001 | NTC |
| FR1P | Ct1 | 19.68 | 19.45 | 19.57 | 30.46 |
| | Ct2 | 20.21 | 19.22 | 19.34 | 36.44 |
| | Ct3 | 19.83 | 19.45 | 19.35 | 35.78 |
| | Ct (average value) | 19.90 | 19.37 | 19.42 | 34.23 |
| FR2P | Ct1 | 26.27 | 25.84 | 26.93 | 44.41 |
| | Ct2 | 26.78 | 25.91 | 27.12 | 43.49 |
| | Ct3 | 26.63 | 26.64 | 27.40 | 43.51 |
| | Ct (average value) | 26.56 | 26.13 | 27.15 | 43.80 |

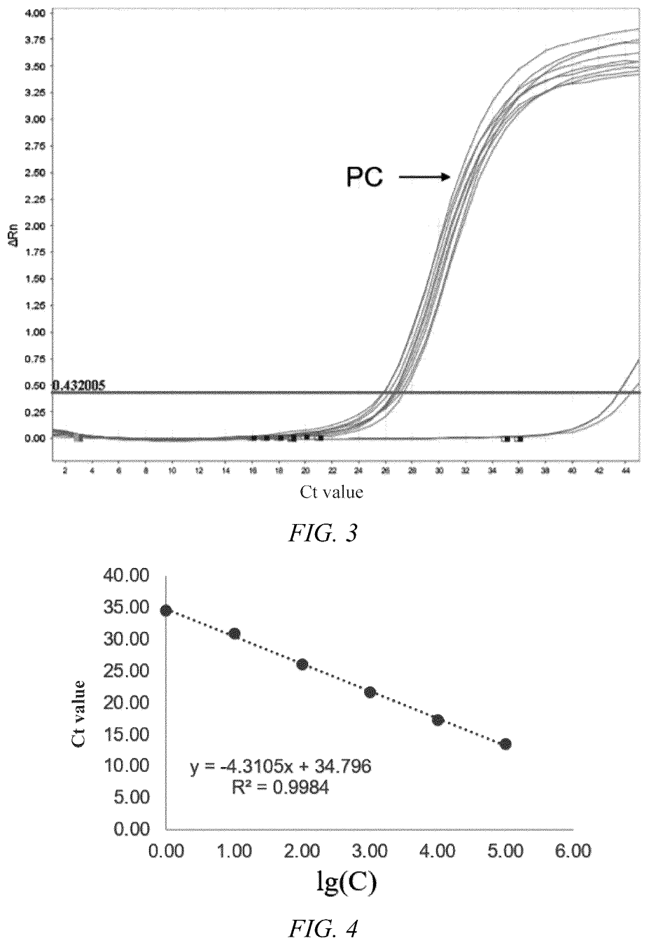

According to the results in Table 3 and the amplification curves corresponding to the FRIP combination and the FR2P combination shown in FIGS. 2 and 3 respectively, it can be seen that the positive controls (PC) of the two groups of primer-probe combinations show normal performance (i.e., the amplification curve is normal and the CT value is within the normal range, for example, less than 35). The Ct value, namely the cycle threshold (Ct), refers to the number of cycles required to amplify a target fragment to a detectable level during RT-PCR detection. The Ct value reflects logarithmic linear correlation with a starting copy number of the sgRNA in each reaction. The larger the Ct value is, the smaller the starting copy number is. When the Ct value is larger than a preset threshold (e.g., 40), it can be considered that the sgRNA is not present in a reaction tube. Therefore, for a negative control, its Ct value should be larger than 40. The average Ct value of the no template control (NTC) for the FR2P combination was 43.80, which was a normal result. However, the average Ct value of the no template control (NTC) for the FRIP combination was 34.23, indicating that false positives would be given when the FRIP combination was used, which would affect ascertainment of the results. Analyzed from a molecular perspective, the R1 primer may non-specifically bind to other sgRNA sequences, resulting in an abnormal Ct value, or form a dimer with the F primer/probe, resulting in an abnormal NTC. Therefore, the primers and probe in the FRIP combination do not have good specificity, and cannot be used. The Ct values of the no-template control (NTC) for the FR2P combination were all larger than 40, satisfying the broad acceptable standard in the field of molecular diagnosis. Therefore, the primer-probe combination FR2P was selected for subsequent detection.

### Real-Time Fluorescence Quantitative PCR

Each components was taken out from a real-time fluorescence quantitative PCR kit (manufacturer: Vazyme, article number: Q223-01), molten on ice (or at room temperature), centrifuged at 1,000 rpm for 15 sec, and then placed in a superclean bench. The volume of each prepared reaction system is as shown in Table 4 below.

TABLE 4
PCR reaction system

| | Loading volume |
|---|---|
| Reaction solution components | (μl)/reaction |
| 2× one-step reaction master mix | 10 |
| One-step enzyme master mix | 1 |
| 50 × ROX dye 2 | 0.4 |
| Primer and probe mix | 1 |
| Nuclease-free water | 2.6 |
| Total volume | 15 |

The use amount of each of the above reagents was computed according to the above Table 4. The reagents were added into a centrifuge tube respectively, fully mixed, and subpackaged into PCR reaction tubes with 15 μl per tube.

5 μl of the sample was added into the PCR reaction tubes prepared above to reach the final reaction volume of 20 μl/tube. The reaction tubes were tightly covered, and centrifuged at 1,000 rpm for 15 sec. The samples were a positive control, a no template control, a collected sample, a reference sgRNA, and a blank control (RNase-free double distilled water), respectively.

Each reaction tube was placed into a real-time fluorescence quantitative PCR instrument to perform PCR amplification according to the procedure in Table 5 below.

TABLE 5
PCR amplification procedure

| | | | Number |
|---|---|---|---|
| Step | Temperature | Time | of cycles |
| Reverse transcription | 55° C. | 15 min | 1 |
| Predenaturation | 95° C. | 30 s | 1 |
| Denaturation | 95° C. | 10 s | 45 |
| Annealing, extension | 60° C. | 30 s (signal collection) | |

Detection fluorescence selection: sgRNA gene (FAM channel), data was collected at 60°° C.

### Sampling Method

The residual sgRNA in the environment was collected by swab sampling. The specific method is as follows:

Sterile nuclease-free water was selected, 500 ul of which was subpackaged into 1.5 ml EP tubes. A sterilized disposable medical swab was first wetted by dipping in 500 ul/tube of water, and then was used to wipe back and forth on a region where a sample was to be collected (such as the floor, desktop, wall surface, shelf, freeze dryer, etc.), that is, a selected preset region, for example, a region with the size of 10 cm×10 cm. After the sampling was complete, the swab was placed into a corresponding EP tube, and broken, and then the tube was covered with the tube cap. At this point, the collection of a sample of local sampling was completed. If the collected to-be-tested sample was not immediately detected, it should be stored in a 4° C. refrigerator.

When completing a production, the production department would perform clearance on the production clean room, focusing on the detection results after clearance, but would also perform collection of a sample of local sampling before clearance, to test the effects of clearance. Therefore, the production clean room before and after clearance was sampled according to the above sampling method.

The accuracy of this sampling method could be validated based on the recycling rate. The specific operations for testing the recycling rate are: first, a known amount of an sgRNA sample was coated on a fixed region, and then a sample of local sampling was collected from the pre-coated region through the above-mentioned swab sampling method. PCR amplification was performed on the collected sample of local sampling, and the Ct value was used to obtain the amount of actually sampled sgRNA. The ratio of the amount of actually sampled sgRNA to the amount of theoretically sampled sgRNA is the recycling rate. The recycling rate data is as shown in Table 6:

TABLE 6
Recycling rate of swab sampling

| | | | Sample | Sample | |
|---|---|---|---|---|---|
| sgRNA | | Ct value | concentration | amount of | |
| sample | Amount of | (average | of actually | actually | |
| concen- | theoretically | of | sampled | sampled | |
| tration | sampled | triplicate | sgRNA | sgRNA | Recycling |
| (pg/ul) | sgRNA (pg) | wells) | (pg/ul) | (pg) | rate |
| 2000 | 20000 | 25.93 | 33.92 | 16957.57 | 84.79% |
| 200 | 2000 | 30.82 | 4.57 | 2287.45 | 114.37% |
| 20 | 200 | 37.26 | 0.33 | 162.66 | 81.33% |

It can be seen from the results in Table 6 that the recycling rate of this sampling method satisfies the range of 50%-150%, which satisfies the acceptance standard for qPCR recycling rate in the 2020 Pharmacopoeia.

### Determination of Standard Curve and Concentration of Sample of Local Sampling

The reference sgRNA containing the universal sequence in FIG. 1 (which is an sgRNA standard at a known concentration of 4,000 ng/ul) was gradiently diluted to obtain a series of diluted samples of ST1-ST6. The concentrations of ST1-ST6 were 100 pg/ul, 10 pg/ul, 1 pg/ul, 0.1 pg/ul, 0.01 pg/ul, and 1 fg/ul respectively. Each concentration was provided with 3 replicates respectively to establish the standard curve. In addition, each collected swab sample (i.e., sample of local sampling) was repeated 3 times. It should be noted that before each loading, the swab sample must be oscillated and centrifuged to ensure that the collected sample of local sampling was distributed in liquid as uniformly as possible, so that the difference between the replicates was smaller, and the results were more credible.

Each swab sample and the reference sgRNA were experimented according to the experimental process of real-time fluorescence quantitative PCR, to obtain the Ct value after detection. According to the Ct value and the concentration of the reference sgRNA, a standard curve was plotted with lg(C) as the abscissa and the Ct value as the ordinate. The standard curve is as shown in FIG. 4.

TABLE 7
lg(C) and Ct values of samples at different concentrations

| Name | Concentration (fg/μL) | lg (C) | Ct value |
|---|---|---|---|
| ST1 | 100000 | 5.00 | 13.54 |
| ST2 | 10000 | 4.00 | 17.29 |
| ST3 | 1000 | 3.00 | 21.64 |
| ST4 | 100 | 2.00 | 26.05 |
| ST5 | 10 | 1.00 | 31.01 |
| ST6 | 1 | 0.00 | 34.60 |

The standard curve is represented by the following formula (3):

$$Y = - 4.3105 X + 34.796 \qquad ( 3 )$$

X=lg(C), C represents the concentration of the collected swab sample, and Y represents the Ct value. The determination coefficient R² of the standard curve is 0.9984, and R²>0.99 means that the standard curve has a good linear relationship. The Ct values of the swab samples were substituted into formula (3) respectively for calculation, to obtain the concentration of each sampled swab sample.

### Determination of Total Residual Amount of sgRNA in Production Environment

Before and after clearance, the determined residual amount of the sgRNA in the production clean room is as shown in Table 7 below. The description is provided with the inner surface-1 of the freeze dryer as an example, the Ct value detected on the inner surface-1 of the freeze dryer is 17.20. The concentration of the sample of local sampling detected on the inner surface-1 of the freeze dryer was computed according to the above-mentioned formula (3). Then, the content of the sgRNA in the EP tube was computed according to the liquid volume in the EP tube being 500 ul based on formula (1). Finally, the residual amount of the sgRNA on the inner surface-1 of the freeze dryer was estimated based on formula (2) according to the content of the sgRNA in the EP tube and the ratio of the swab sampling area S1 to the area S2 of the inner surface-1 of the freeze dryer. The detection and computing method for the residual amount of the sgRNA in other regions was the same as above. 5 Finally, all residual amounts of the sgRNA were summed to obtain the total residual amount of the sgRNA in the production environment.

TABLE 8
Detection results before and after clearance
Highest value data

| | Before clearance | | After clearance | |
|---|---|---|---|---|
| | | Residual | | Residual |
| | Average | amount | Average | amount |
| | value | of sgRNA | value | of sgRNA |
| Sampling position | of Ct | (ng) | of Ct | (ng) |
| #1-wall surface | 25.22 | 117.98 | 30.07 | 8.90 |
| #2-wall surface | 21.05 | 921.96 | 29.98 | 21.30 |
| #3-wall surface | 27.99 | 33.36 | 28.78 | 17.84 |
| #4-wall surface | 29.31 | 12.81 | 27.93 | 98.84 |
| #5-roof | 21.50 | 1765.21 | 32.14 | 20.51 |
| #6-floor | 22.65 | 917.80 | 29.04 | 1077.43 |
| #7-wall surface | 27.65 | 5.09 | 30.15 | 4.08 |
| #8-wall surface | 24.26 | 54.80 | 25.18 | 15.68 |
| Inner surface-1 of | 17.20 | 122.11 | 20.85 | 13.30 |
| freeze dryer | | | | |
| Inner surface-2 of | 20.94 | 23.81 | 20.83 | 13.46 |
| freeze dryer | | | | |
| Inner surface-3 of | 16.19 | 354.42 | 19.93 | 28.44 |
| freeze dryer | | | | |
| Inner surface-4 of | 12.68 | 2110.48 | 22.10 | 9.39 |
| freeze dryer | | | | |
| Inner surface-5 of | 19.99 | 42.80 | 20.58 | 17.01 |
| freeze dryer | | | | |
| Inner surface-6 of | 21.94 | 15.91 | 16.50 | 197.90 |
| freeze dryer | | | | |
| Pallet 1-upper surface | 16.82 | 154.64 | 16.24 | 163.43 |
| Pallet 1-lower surface | 10.85 | 3210.15 | 28.35 | 0.25 |
| Pallet 2-upper surface | 12.03 | 1759.10 | 12.98 | 932.43 |
| Pallet 2-lower surface | 16.39 | 192.42 | 28.37 | 0.25 |
| Total | | 11.81 | | 2.64 |

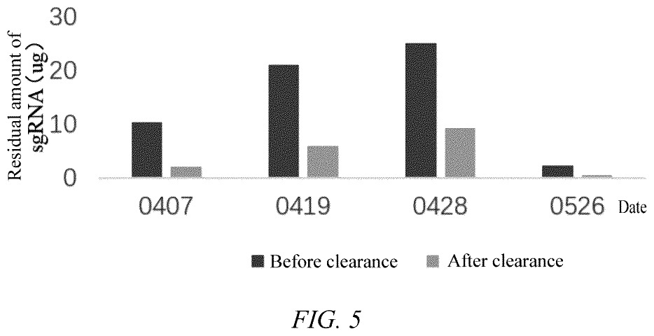

FIG. 5 shows a diagram of comparison among total residual amount results of an sgRNA in an environment before and after clearance at different stages (The abscissa is the date of detection, and the ordinate is the residual amount of the sgRNA, unit: ug). Table 8 shows the data of the particularly detected residual amount of the sgRNA in each region dated April 7 in FIG. 5. It can be seen from the results in FIG. 5 and Table 8 that after each clearance, the residual amount of the sgRNA is significantly reduced compared to that before clearance, indicating that clearance has significantly improved the reduction of residues. In each production scale of 20 mg, it was found after testing that, when the residual amount of the sgRNA was less than 20 ug (one thousandth), it had basically no impact on downstream biological applications. The results showed that the residual amount after each clearance was far less than the threshold of one thousandth, indicating that the sgRNA had basically no impact on the downstream applications.

A primer, a kit and a method for detecting the residual amount of sgRNA in a environment disclosed in the present application have brought beneficial effects, including but not limited to: (1) the primer pair and the probe involved for the universal sgRNA sequence have good specificity, high sensitivity and high repeatability; and (2) the real-time fluorescent quantitative PCR technology based on fluorescent probes is used to detect the residual amount of the sgRNA in the environment, and perform real-time and high-throughput detection of the residual amount of the sgRNA in the environment at different stages of the production process, and is characterized by convenient operations, high sensitivity, low costs, short cycle, and high accuracy. It should be noted that different embodiments may have different beneficial effects, and the beneficial effects that may be produced in the different embodiments may be any one or a combination of the above, or may be any other beneficial effects that may be obtained.

It should be understood by those skilled in the art that the above embodiments are only for illustrating the present invention, and do not limit the present invention. Any modifications, equivalent substitutions, changes, and the like made within the spirit and principle of the present invention should fall within the scope of protection of the present invention.

1. A primer pair for detecting a residual amount of a small guide RNA (sgRNA) in an environment, wherein a forward primer of the primer pair is a nucleic acid molecule as shown in SEQ ID NO: 2, and a reverse primer of the primer pair is a nucleic acid molecule as shown in SEQ ID NO: 5.

2. The primer pair according to claim 1, wherein the sgRNA comprises a sequence as shown in SEQ ID NO: 1.

3. A kit for detecting a residual amount of an sgRNA in an environment, comprising the primer pair according to claim 1.

4. The kit according to claim 3, further comprising a fluorescent probe, the fluorescent probe is a nucleic acid molecule as shown in SEQ ID NO: 4 with a fluorescent group connected at 5′-terminal of the nucleic acid molecule and a quenching group connected at 3′-terminal of the nucleic acid molecule.

5. The kit according to claim 4, wherein
the fluorescent group comprises FAM, JOE, JA270, TET, Cal Fluor Gold 540, HEX, VIC, Cal Fluor Orang 560, TAMRA, Cyanine 3, Quasar 570, Cal Fluor Red 590, Rox, Texas Red, Cyanine 5, Quasar 670, or Cyanine 5.5; and
the quenching group comprises MGB, TAMRA, DABCYL, BHQI-3, or Eclipse.

6. The kit according to claim 4, wherein the sgRNA comprises a sequence as shown in SEQ ID NO: 1.

7. The kit according to claim 3, wherein the kit is used in a real-time fluorescence quantitative PCR system.

8. A method for detecting a residual amount of an sgRNA in an environment, comprising:
1. designing a primer pair and a fluorescent probe based on a sequence of a residual sgRNA in the environment;
2. locally sampling a region in the environment to obtain a sample;
3. performing real-time fluorescence quantitative PCR on the sample obtained from the local sampling using the primer pair and the probe in step 1), and determining a concentration of the sgRNA in the sample obtained from the local sampling using a Ct value; and
4. determining the residual amount of the sgRNA in the environment based on the concentration of the sgRNA in the sample determined in step 3).

9. The method according to claim 8, wherein the sgRNA comprises a sequence as shown in SEQ ID NO: 1.

10. The method according to claim 8, wherein a forward primer of the primer pair is a nucleic acid sequence as shown in SEQ ID NO: 2, and a reverse primer of the primer pair is a nucleic acid sequence as shown in SEQ ID NO: 5.

11. The method according to claim 8, wherein the fluorescent probe is a nucleic acid molecule with a fluorescent group connected at 5′-terminal of the nucleic acid molecule and a quenching group connected at 3′-terminal of the nucleic acid molecule.

12. The method according to claim 8, wherein the fluorescent probe comprises a nucleic acid molecule as shown in SEQ ID NO: 4.

13. The method according to claim 8, wherein the determining the residual amount of the sgRNA in the environment based on the concentration of the sgRNA in the sample determined in step 3) comprises:
determining a content of the sgRNA in the sample based on the concentration of the sgRNA in the sample; and
determining the residual amount of the sgRNA in the environment based on the content of the sgRNA in the sample.

14. The method according to claim 11, wherein
the fluorescent group comprises FAM, JOE, JA270, TET, Cal Fluor Gold 540, HEX, VIC, Cal Fluor Orang 560, TAMRA, Cyanine 3, Quasar 570, Cal Fluor Red 590, Rox, Texas Red, Cyanine 5, Quasar 670, or Cyanine 5.5; and
the quenching group comprises MGB, TAMRA, DABCYL, BHQI-3, or Eclipse.
